(19) United States
(12) Patent Application Publication
(10) Pub. No.: US 2019/0145947 A1
(43) Pub. Date: May 16, 2019

(54) Method and Device for Estimation of Alcohol Content in Fermentation or Distillation Vessels

(71) Applicant: Wil McCarthy, (US)

(72) Inventors: Chris Oltyan, Lakewood, CO (US); Wil McCarthy, Lakewood, CO (US)

(21) Appl. No.: 16/170,995

(22) Filed: Oct. 25, 2018

Related U.S. Application Data
(60) Provisional application No. 62/584,781, filed on Nov. 11, 2017.

(51) Int. Cl. G01N 33/14; G01N 35/00; G01N 1/22; G01N 33/00

(52) U.S. Cl. CPC G01N 33/146; G01N 35/00871; G01N 1/2226; G01N 2001/2229; G01N 33/0044; G01N 33/0049; G01N 2035/00346; G01N 33/004

(57) ABSTRACT

The subject matter described herein relates to a device and method for estimating the alcohol-by-volume (ABV) of a liquid inside a fermentation or distillation vessel, without opening the vessel or requiring a liquid sample. Other properties of the liquid may also be estimated using this method, by including additional sensors in the device. This method has particular, but not exclusive, application in the home brew, microbrew, home and small batch winemaking, and small-batch distillery industries.

### CROSS REFERENCE TO RELATED APPLICATIONS

This application claims the priority benefit of U.S. Provisional Patent Application No. 62/584,781 filed 11 Nov. 2017 for Chris Oltyan and Wil McCarthy, hereby incorporated by reference in its entirety as though fully set forth herein.

### BACKGROUND

### 1. Technical Field

The subject matter described herein relates to a method for estimating the alcohol content of a liquid in a fermentation or distillation vessel, without opening the vessel and without touching the liquid.

### 2. Description of the Related Art

In the beer, wine, and liquor industry, including the microbrew industry as well as specialty products such as kombucha, it is a legal requirement that products of a given title, label, and recipe provide a consistent alcohol-by-volume (ABV). Furthermore, even in amateur and hobby crafts such as homebrewing and winemaking, the desire for a consistent product is widespread.

In the existing art, this need is most often met through the use of a hydrometer, which measures the specific gravity of the liquid, i.e., the density of the liquid divided by the density of pure water. Sugars are heavier than water, and alcohol is lighter than water, so as sugar molecules are converted into alcohol molecules through the fermentation or distillation process, the density of the liquid decreases. However, this method requires the withdrawal of a fairly large quantity of liquid from the fermentation vessel.

To address this problem, new methods and devices were developed that use the index of refraction of the liquid to estimate alcohol content. This requires a much smaller sample, but still requires the vessel to be opened and liquid to be withdrawn from it. Also, it should be noted that a refractometer does not directly measure specific gravity. With knowledge of other variables such as temperature, this reading may be formulaically converted into a specific gravity value. Importantly, both instruments require physical contact with a sample of the liquid that has been removed from the fermentation or distillation vessel. This is not desirable, since opening the vessel during the fermentation or distillation process increases the possibility for contamination, and removing daily samples reduces the volume of salable product yielded by the process.

Furthermore, neither measurement can be converted into an ABV value unless the starting value of the mix, prior to the start of fermentation or distillation, is known. Instead, the change in specific gravity is used to estimate the mass of sugar that has been converted into alcohol, and thus an alcohol-by-weight estimate. This is then converted into ABV. If the starting value is not known or is not recorded correctly, then accurate calculation of ABV is not possible. In addition, neither method accounts accurately for solids in the vessel that are not dissolved or suspended at the time of measurement, even where such solids may eventually contribute to alcohol content.

U.S. Pat. No. 9,034,171 to Mitchell et. al. discloses a system for monitoring the fermentation process of a liquid, involving a fuel-cell sensor to detect alcohol vapor in the headspace gas of a fermentation vat. Mitchell states that the concentration of alcohol in the gas is proportional to the concentration of alcohol in the liquid, which is a significant oversimplification lacking enablement. In addition, fuel-cell alcohol sensors (being intended for use in breathalyzers) are generally extremely sensitive, and their readings vary with significant changes in the temperature or humidity of the measured gas, and they are subject to false-positive contamination by normal fermentation byproducts such as acetic acid. Thus, their use in a fermentation vessel without a permanently “maxed out” or otherwise inaccurate reading would require further inventive steps in order to make them function reliably in the manner Mitchell describes. Essentially, the “calibration” procedure described by Mitchell in Column 7, Paragraph 4 of his patent would be the calibration for a particular fermentation mix at a particular gas temperature and relative humidity. If any of these conditions changed significantly, the calibration would be invalidated and the measurements would be inaccurate.

Given the lack of enabling detail in Mitchell's disclosure, it is not clear that Mitchell was fully in possession of the invention he claimed at the time of his filing. Nevertheless, we assert that Mitchell's claimed invention is also flawed, in that it requires “the originally determined specific gravity of the liquid prior to fermentation to ascertain the progression and development of the fermentation over time.” Since the specific gravity of the starting solution must be known, the method cannot properly be described as touch-free, and would address only part of the measurement burden over previously existing technologies. More importantly, although Mitchell's specification touches briefly (and without enabling detail) on the issue of relating the ABV of the gas to the ABV of the liquid, none of Mitchell's claims actually recite this step. His Claim 6 does state that the alcohol gas measurement can be related to a specific gravity, but this would still not eliminate the need for users to perform additional calculations in order to generate an ABV estimate from the specific gravity described in the claim. Thus, while Mitchell's claimed invention may indeed “monitor fermentation”, it does not calculate the ABV of a liquid and thus is not responsive to the industry's need.

The previously existing related art does not include a practical, touch-free method for directly measuring and reporting the estimated ABV of a liquid without opening the fermentation or distillation vessel and removing samples from it, and without performing additional hand calculations. Such a sample-free, hand-calculation-free method is highly desirable in that ABV readings could be obtained frequently or even continuously, without the inconvenience of opening the fermentation or distillation vessel, without the risk of contamination while the vessel is open, without the loss of sampled material from the volume of final product, and without the need to perform additional calculations or the chance of performing said calculations incorrectly. Such a method is clearly desirable, both for the home hobbyist and for the professional brewer, vintner, fermenter or distiller, and represents a long-felt but unsolved need within these industries, that other practitioners have failed to address.

The information included in this Background section of the specification, including any references cited herein and any description or discussion thereof, is included for technical reference purposes only and is not to be regarded as subject matter by which the scope of the invention is to be bound.

### SUMMARY

Disclosed is a method and device for the estimation of alcohol content in fermentation or distillation vessels. A gas sample is taken from the fermentation or distillation vessel, either at the airlock, at a pouring valve, or at any other convenient location that does not require the vessel to be opened. In an example, the method also does not require any modifications to the vessel. From this gas sample, three variables are measured: the temperature and humidity of the gas, along with its alcohol content. These three variables are then used to compute an ABV estimate of the liquid inside the vessel.

Alcohol content may be measured using an alcohol gas sensor. In an example, the invention employs an alcohol gas sensor conforming to the MQ-3 specification, due to its low cost and solid-state design, although a variety of other sensors may be used instead (including the fuel cell sensors described by Mitchell, although these are not preferred). In an MQ-3 alcohol gas sensor, a semiconductor part is heated, whose resistance varies as alcohol molecules adsorb onto its surface. The semiconductor also responds to other molecules, such as carbon monoxide and hydrogen gas, but is significantly more sensitive to alcohol, such that the influence of other molecules can generally be neglected, with the exception of water vapor. When a semiconductor-based alcohol sensor is first activated, there are two sources of sensor lag: a first delay while the sensor is heated to an equilibrium temperature, and a second delay while the amount of adsorbed alcohol settles to an equilibrium level. These lags are not taught in the related art.

Once sensor equilibrium is achieved, the resistance of the MQ-3 sensor (or other semiconductor-based alcohol sensor) is a function of the temperature, humidity, and alcohol content of the gas. In other words, the actual alcohol content is not measured, but may be deduced if the temperature and humidity are known (as in the case of human breath) or are not relevant (as in the case of factory air being “sniffed” by an alarm sensor for the presence of trace alcohol gas). This is true, to varying degrees, regardless of the exact type of alcohol sensor employed.

Therefore, in the method and device of the present disclosure a temperature and humidity sensor are required, and represent a clear improvement over the related art. These come in a variety of different types, and may be two different components or may be combined into a single unit. In an example, a combined temperature and humidity sensor conforming to the DHT-22 (also known as RHT-03, AM2302, or ADA393) standard is employed. This sensor reports temperature and humidity in a digital format over a serial interface.

In order for sensors of this type or any similar type to be read, the device must include both a power supply and a microprocessor. In an example, the microprocessor reads the digital or analog values coming from the temperature and humidity sensor or sensors. In an example, the microprocessor may also perform the calculation of ABV based on a three-variables-in/one-variable out regression, and may optionally report this value to an external processor such as an app running on a computer, tablet, mobile phone, or remote server. Alternatively, the sensor values themselves may be reported to an external processor, and an ABV value may be calculated there, either using stored regression parameters or else via machine learning techniques that analyze a large set of four-variable data points (alcohol sensor reading, temperature, humidity, and the corresponding ABV from a known sample) and compute the ABV as a “most likely” output variable. It is also possible for machine learning to be employed directly on the microprocessor, and such a configuration is explicitly claimed as part of the present disclosure.

Where such reporting to an outside processor occurs (either of the sensor values or of the computed ABV estimate), it may be through a variety of different methods, including but not limited to Bluetooth, WiFi, or other wireless communication protocol, or any of a multitude of wired interfaces including but not limited to RS-232, Ethernet, and USB. Alternatively, the device may report an ABV estimate directly to the user, via a video display, a digital meter, an analog meter, a series of progressive LEDs with values marked next to them, or other related methods.

The device of the present disclosure consists of six basic elements: a power source, an alcohol gas sensor, a temperature sensor, a humidity sensor, a microprocessor, and a means of reporting, displaying, or transmitting measured or computed values as described above. Optionally, it may also include external elements such as an app for receiving sensor or ABV values, calculating ABV values based on transmitted sensor readings, and displaying an ABV estimate either numerically, graphically, comparatively, or in some other convenient form such as vibration or audio tones.

The device may also accommodate the addition of other sensors that can capture information from the solution being measured and relay that information to microprocessing units. These sensors may capture additional characteristics of the gas, or may contain probes that extend into the solution being measured.

The descriptions in this summary are intended to be illustrative and/or exemplary, and not limiting.

### BRIEF DESCRIPTION OF THE DRAWINGS

FIG. 1 is a graph of the steady-state resistivity of an MQ-3 compliant sensor as a function of gas concentration, for three different gases.

FIG. 2 is a graph of the steady-state resistivity of an MQ-3 compliant sensor as a function of temperature and humidity.

FIG. 3 is a schematic representation of an example device for estimating ABV, including all components and wiring.

FIG. 4 is an exemplary regression curve matching the alcohol gas sensor reading to an estimated ABV value.

FIG. 5 is a drawing of an exemplary implementation of the present invention while in use.

### DETAILED DESCRIPTION

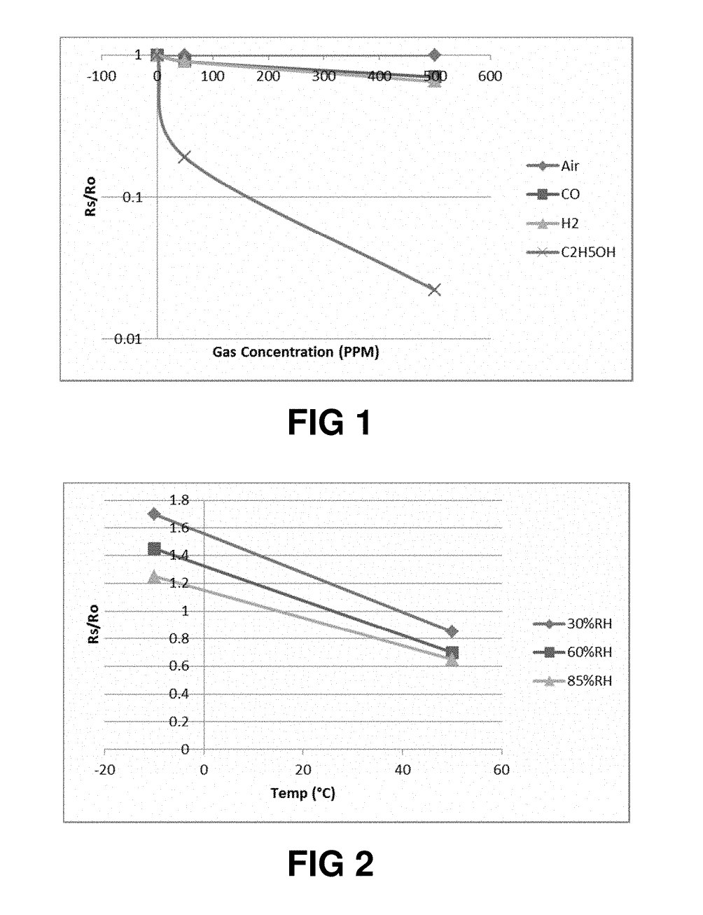

FIG. 1 is from the related art, and is a graph of the steady-state resistivity of an MQ-3 compliant sensor as a function of gas concentration, for three different gases. The graph shows that the sensor is significantly more sensitive to alcohol than to carbon monoxide or hydrogen, such that the presence or absence of either of these two gases may reasonably be ignored without significant effect on the measured value. However, the sensitivity to alcohol is significant, such that the device can be used to detect alcohol vapor that rises up from the liquid in a fermentation or distillation vessel.

FIG. 2 is also from the related art, and is a graph of the steady-state resistivity of an MQ-3 compliant sensor as a function of temperature and humidity. The graph shows that the readings of the alcohol gas sensor are significantly affected by both temperature and humidity, and that an alcohol vapor concentration measurement based on the readings of the alcohol sensor alone will be highly inaccurate unless the temperature and humidity can be assumed to hold fixed values (as is the case with human breath). Conversely, the graph shows that if the temperature and humidity are known accurately, that the readings of an MQ-3 compliant alcohol gas sensor can be used to obtain an accurate alcohol vapor concentration. The same principle can be applied to other alcohol sensors, which may be similarly sensitive to moisture and temperature, though not necessarily at the same ratios.

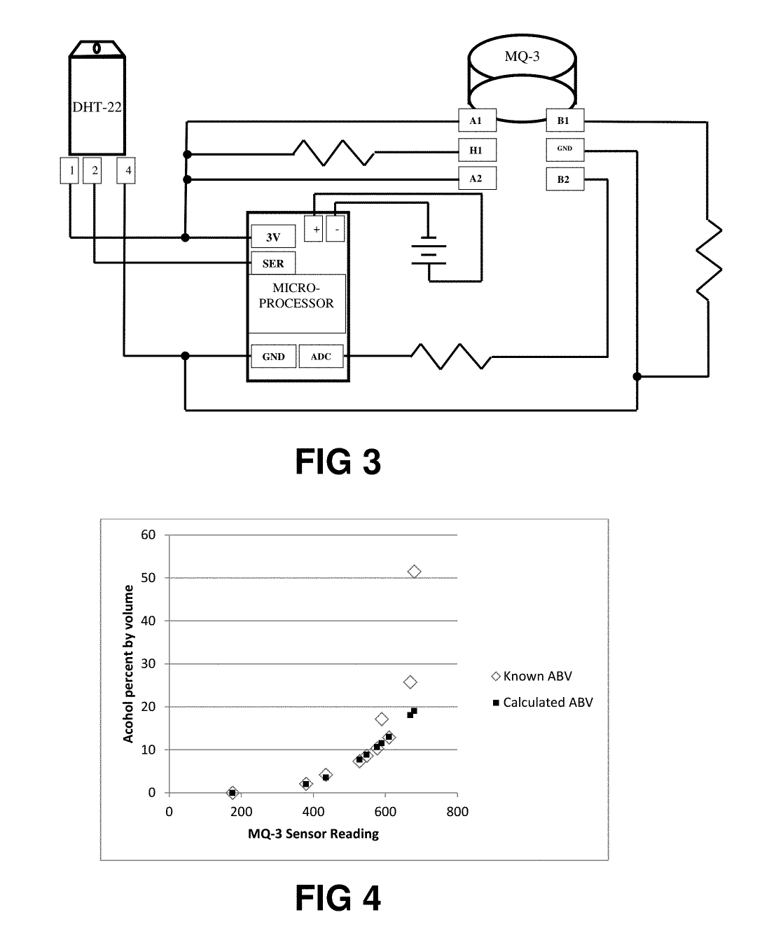

FIG. 3 is is a schematic representation of an exemple touchless device for estimating ABV in a fermentation or distillation vessel, including all components and wiring. A DHT-22 temperature and humidity sensor is powered from a 3.3V source on the microprocessor, and reports its readings through a serial connection on the microprocessor. The MQ-3 alcohol gas vapor sensor receives heater power and sensor power from the same or a similar 3.3V source, and outputs an analog voltage that is read by an analog input on the microprocessor. Scaling resistors are included on certain signal and ground lines so that the input values can be tuned to the voltage expectations of the microprocessor.

In addition to scaling resistors, further steps may be required to adjust the output of the alcohol gas sensor so that it produces signals that the processor can interpret. When employed in breathalyzer sensors to determine blood alcohol content of an individual, an alcohol sensor is detecting very small quantities in the range of 0.02-0.16% alcohol within the bloodstream, and substantially smaller quantities in the exhaled breath of an individual that equate to these values. Similarly, when used in alcohol vapor alarm systems, the alcohol gas sensor is essentially indicating whether alcohol vapor is present in the air in trace concentrations, and is generally used to report either a “yes” (alcohol vapor is present) or a “no” (alcohol vapor is not present or is below detectable concentrations). In neither case is the temperature or humidity of the gas relevant; exhaled human breath is always close to body temperature and always close to 100% relative humidity, and minor variation around these values does not have a significant effect on the alcohol concentration reading. In the case of an alarm sensor, the only question may be whether the sensor detects alcohol vapor at all; the exact concentration may be irrelevant. Thus, there is no need in either case to measure temperature or humidity, and the related art does not teach the value of such measurements as a means of detecting alcohol accurately in fermentation or distillation vessels.

However, in the case of both breathalyzer and alcohol alarm sensors, the sensor (e.g., an MQ-3 compliant sensor) needs to be extremely sensitive in order to perform its intended function. Where such low-cost, commodity sensors are employed in the device of the present disclosure, they may be so sensitive that in the environment of use (e.g., the airlock of a fermentation vessel containing a liquid of 2-12% ABV or a distillation vessel containing a liquid of 20-90% ABV), they consistently send out a signal that the microprocessor will interpret as “maximum” or “infinity”. Thus, it may be necessary not only to scale the sensor's output (e.g., by adding resistors to limit its input and output voltages), but also to “starve” its heater circuit by providing it with less than the specified 3.3V input voltage. This results in a lower heater temperature and thus less sensitivity for the sensor itself—something that is not taught in the related art and would not be known to a practitioner of the art, nor discovered by said practitioner without both an inventive step and considerable experimentation.

In addition to “starving” the heater circuit, it is possible to clean the surface of the semiconductor portions of the sensor in order to desorb molecules from its surface. This is done by increasing its supply voltage to 5V or higher, such that its operating temperature (while remaining within safe limits) increases for the duration of the cleaning cycle.

FIG. 4 is an example regression curve matching the alcohol gas sensor reading to an estimated ABV value for a particular temperature and humidity. As the graph shows, it may be difficult to define a single function that accurately describes the sensor response curve across all possible ABV values. In the case shown, the equation:

ABV=((ADC reading−148.0)̂2.7)/1.2E6

results in a curve that accurately fits the known ABV values, for alcohol sensor readings between 200 and 600 counts (arbitrary units set by the scaling resistors). However, for values below 200 counts, the curve is simply equal to zero, and for values above 600 counts it clearly follows a different shape, and may in fact be most accurately represented by a steep, straight line. These calibration curves will be unique for each sensor type, heater power level, and set of scaling resistor values. The curve fit or machine learning calculations which apply these curves may be performed either onboard the microprocessor itself, or else on a remote processor such as a mobile phone, tablet or laptop computer, or server.

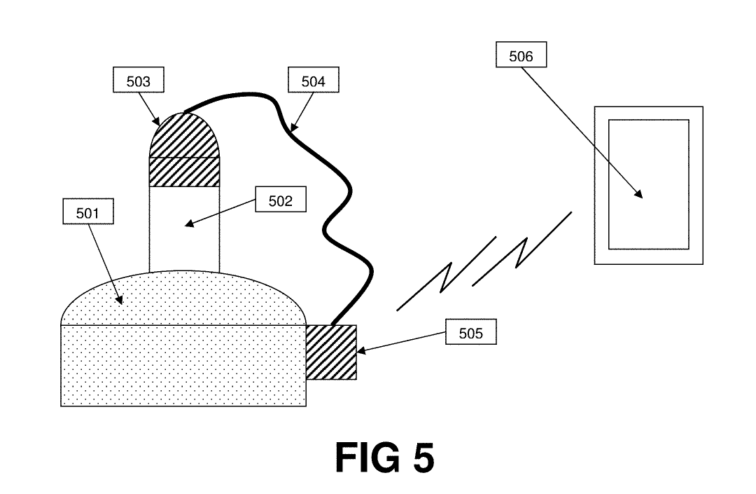

FIG. 5 is a drawing of an exemplary implementation of the present invention while in use. The fermenting liquid (not shown) is enclosed inside a fermentation or distillation vessel 501, which includes an airlock 502 that prevents pressure buildup by allowing gases such as carbon dioxide to escape during the fermentation or distillation process. Normally, the airlock would include a porous plastic cap, but in this case the cap has been replaced with a sensor head 503 that contains the temperature, humidity, and alcohol vapor sensors. In an example, the sensor head is a flexible rubber end cap that fits snugly over the top of the airlock, and may or may not include holes for gas to escape, other than a through-hole for a cable 504 to pass through, which may or may not include an airtight seal. The cable 504 connects the sensor head to the electronics enclosure 505, providing power, ground, and communications as shown for exemplary purposes in the circuit diagram of FIG. 3. In a preferred embodiment, the cable includes connectors such that equivalent cables of different lengths can readily be employed, to match the geometry of the fermentation or distillation environment with as much convenience as possible. The electronics enclosure 505 includes the microprocessor, plus any resistors, capacitors, wiring, power sources or power connectors, and supporting hardware such as cooling fans that may be necessary for its correct operation. The electronics enclosure may be attached to, suspended from, or adjacent to the fermentation or distillation vessel, or may (with a long enough cable) be located some distance away.

The electronic enclosure also includes either a means to display a calculated ABV estimate for the liquid inside the fermentation or distillation vessel 501, or else a means to transmit the calculated ABV value, or the raw sensor values, to an optional remote processor 506, such as a mobile phone, tablet or laptop computer, or server. The remote processor 506 may compute the ABV directly, or it may receive the ABV estimate from the microprocessor within the electronics enclosure 505. In either case, the remote processor is capable of either displaying the ABV (whether graphically, numerically, comparatively, or in some other form), or else posting values to a web page, database, or other medium from which it may be retrieved through a variety of means that will be familiar to a person skilled in the art, and need not be reiterated here.

Communication between the microprocessor within the electronics enclosure 505, and the optional remote processor 506, may be through Bluetooth, WiFi, or some other wireless communication protocol, or it may occur through a wired connection such as a USB cable. The remote processor may be contacted directly, or via a local area network, or over the Internet or some other wide-area network.

Numerous variations on the disclosed embodiments are also possible, by means of deleting or combining certain components. For example, the sensor head may not be connected to an airlock, but to a tap, drain valve, inspection port, or any other aperture in the fermentation or distillation vessel. Indeed, different industries and different vessel types may each have their own preferred location for the sensors, with the shape of the sensor head being optimized to accommodate such locations. The sensor head could even be placed on a container (e.g., a bottle) of fully-fermented product such as a bottle of wine, beer, kombucha, or liquor, to verify its ABV. This could be done for quality control purposes, or as part of an inventory control process (e.g., to determine whether liquor had been watered down to disguise a theft of material from a bar). Also, for example, by studying the readings of an alcohol gas sensor during its heater warmup and sensor settling periods, a machine learning algorithm may deduce the temperature and humidity of the gas inside the sensor head, thus serving as a “virtual” temperature and humidity sensor.

In addition, components may be added to, or substituted for, those shown in the Figures. For example, a CO2, CO, H2S, or Ethene/Ethylene gas sensor may be substituted for, or included alongside, the alcohol gas sensor. Such sensors are available that are closely related to the MQ-3 alcohol sensor, and work on the same general principle, although sensors of other types may be employed as well, without departing from the spirit of the present disclosure. The use of additional gas sensors would enable the measurement or estimation of other properties of the liquid such as sourness, bitterness, “skunkiness”, or contamination. Alternatively or in addition, the device could include food-grade electrical resistance probes which extend from the microprocessor in the electronics housing, through an aperture in the fermentation or distillation vessel, and directly into the liquid. This would enable a measurement of the electrical resistance of the liquid, which correlates to alcohol content, sugar content, and suspended solids content. With machine learning algorithms or other algorithms employing a large data set of readings from different alcoholic beverages in various states of fermentation or distillation, it is then possible to calculate or estimate the correlation of each sensor value to different chemical, olfactory, or flavor components of the liquid, and thus with the totality of measurements to deduce a great deal about the chemistry of the liquid, and thus produce a predicted or estimated flavor profile including sweet, sour, bitter, astringent, skunky, spoiled, and umani flavors, as well as ABV.

In addition, the components of the present disclosure may be formulated from different materials or in different forms than those disclosed herein, so long as they perform an equivalent physical or chemical function. For example, the electronics housing may take on an artistic or aesthetic form, or may be absent altogether, such that the microprocessor may be directly exposed to the sight of users and passersby. The sensor head could take a number of different physical forms, including no sensor head enclosure at all, but simply the sensors themselves (which could be clipped into place near an aperture in the fermentation or distillation vessel). Such variations do not depart from the spirit of the present disclosure.

Thus, a reader of ordinary skill in the art will understand that the present disclosure encompasses a variety of dissimilar but functionally equivalent methods and device designs. The above specification, examples and data provide a description of the structure and use of some exemplary embodiments of the invention. Although various embodiments of the invention have been described above with a certain degree of particularity, or with reference to one or more individual embodiments, those skilled in the art could make numerous alterations to the disclosed embodiments without departing from the spirit or scope of this invention. Other embodiments are therefore contemplated. All directional references e.g., proximal, distal, upper, lower, inner, outer, upward, downward, left, right, lateral, front, back, top, bottom, above, below, vertical, horizontal, clockwise, and counterclockwise are only used for identification purposes to aid the reader's understanding of the present invention, and do not create limitations, particularly as to the position, orientation, or use of the invention. Connection references, e.g., attached, coupled, connected, and joined are to be construed broadly and may include intermediate members between a collection of elements and relative movement between elements unless otherwise indicated. As such, connection references do not necessarily imply that two elements are directly connected and in fixed relation to each other. Changes in detail or structure may be made without departing from the basic elements of the invention as defined in the following claims.

What is claimed is:

1. A method for estimating the alcohol-by-volume of a liquid in a fermentation or distillation vessel, or in a container of fermented or distilled liquid, comprising:
sampling the gas within the vessel or through an aperture within the vessel;
waiting until all sensors have settled into a steady state;
measuring the temperature, humidity, and alcohol concentration of the gas;
comparing the sensor readings against curve fits or machine learning algorithms based on data sets of known ABV, temperature, and humidity, to compute an estimated ABV for the liquid and;
either storing the estimated ABV, reporting it to a user, reporting it to an external processor, uploading it to a remote site, or any combination thereof.

2. The method of claim 1 wherein the alcohol concentration of the gas is measured with a semiconductor based alcohol selected from a set that includes but is not limited to MQ-3 compliant sensors.

3. The method of claim 1 wherein the temperature and humidity of the gas are measured with a DHT-22 compliant sensor.

4. The method of claim 1 wherein the output of the alcohol sensor is scaled to match the expectations of the microprocessor.

5. The method of claim 1, wherein the sensitivity of the alcohol sensor is reduced from its native or expected state.

6. The method of claim 1, wherein the sensor is cleaned automatically by means of a cleaning cycle.

7. The method of claim 6, wherein the cleaning cycle is achieved through a voltage increase to a heater circuit.

8. The method of claim 1, wherein the aperture is a carboy airlock.

9. A device for estimating the alcohol-by-volume of a liquid inside a fermentation or distillation vessel, or in a container of fermented or distilled liquid, comprising:
an alcohol gas sensor, temperature sensor, humidity sensor, microprocessor, power supply and;
all necessary wires, resistors, and firmware needed to connect these elements in a functional manner and;
an algorithm for comparing sensor readings against legacy data sets for known ABV to produce an estimate of the ABV of the liquid from a sample of the gas emitted by it, and;
a means of reporting, transmitting, displaying, or storing the estimate of the ABV.

10. The device of claim 9 wherein the resistors include one or more scaling resistors to match the output of the alcohol sensor to the voltage expectations of the microprocessor.

11. The device of claim 9 wherein the resistors include one or more “starving” resistors to reduce the voltage to a heater circuit and thus decrease the sensitivity of the alcohol sensor.

12. The device of claim 9 wherein the alcohol sensor can be cleaned by applying a higher-than-expected voltage to its heating circuit for a period of time.

13. The device of claim 9, wherein device is attached to a carboy airlock.

14. The device of claim 9 wherein other or additional gas sensors are employed to:
Measure any or all of the CO2, CO, H2S, and Ethene/Ethylene gas concentrations emitted by the liquid and,
additional algorithms are employed to relate these measurements to other properties of the liquid such as sweetness, bitterness, skunkiness, astringency, spoilage, and umami.

15. The device of claim 9 wherein food-grade electrical resistance probes are immersed in the liquid in order to measure its electrical resistivity and,
additional algorithms are employed to relate this measurement to ABV and other properties of the liquid such as sweetness, bitterness, skunkiness, astringency, and umami.

16. A system for estimating the alcohol-by-volume of a liquid inside a fermentation or distillation vessel, or in a container of fermented or distilled liquid, comprising:
an alcohol gas sensor, temperature sensor, humidity sensor, microprocessor, power supply and;
all necessary wires, resistors, and firmware needed to connect these elements in a functional manner and;
an algorithm for comparing sensor readings against legacy data sets for known ABV to produce an estimate of the ABV of the liquid from a sample of the gas emitted by it, and;
a means of reporting, transmitting, displaying, or storing the estimate of the ABV.

17. The system of claim 16 further comprising any or all of:
one or more scaling resistors to match the output of the alcohol sensor with the expectations of the microprocessor;
one or more “starving” resistors to reduce the voltage to a heater circuit and thus decrease the sensitivity of the alcohol sensor.

18. The system of claim 16 wherein the alcohol sensor can be cleaned by applying a higher-than-expected voltage to its heating circuit for a period of time.

19. The system of claim 16, wherein device is attached to a carboy airlock.

20. The system of claim 16, additionally comprising either or both of:
additional gas sensors are employed to measure any or all of the CO2, CO, H2S, and Ethene/Ethylene gas concentrations emitted by the liquid, or;
food-grade electrical resistance probes immersed in the liquid in order to measure its electrical resistivity, and;
algorithms to relate the measurements to other properties of the liquid such as sweetness, bitterness, skunkiness, astringency, spoilage, and umami.
